(19) United States
(12) Patent Application Publication
(10) Pub. No.: US 2015/0342961 A1
(43) Pub. Date: Dec. 3, 2015

(54) USE OF PROSTAGLANDIN E1 (PGE1) AND MISOPROSTOL FOR TREATING CHRONIC MYELOGENOUS/MYELOID LEUKEMIA (CML)

(71) Applicant: Hai-Hui Xue, Iowa City, IA (US)

(72) Inventor: Hai-Hui Xue, Iowa City, IA (US)

(21) Appl. No.: 14/729,937

(22) Filed: Jun. 3, 2015

Related U.S. Application Data
(60) Provisional application No. 62/007,243, filed on Jun. 3, 2014.

(51) Int. Cl. A61K 31/5575; A61K 31/506

(52) U.S. Cl. CPC A61K 31/5575; A61K 31/506

(57) ABSTRACT

Disclosed are methods and compositions for treating a patient having chronic myelogenous/myeloid leukemia (CML). The methods utilize and the compositions include prostaglandin E1 (PGE1) or Misoprotol.

### BACKGROUND

The field of the invention relates to methods of treating patients having chronic myelogenous/myeloid leukemia (CML). In particular, the field of the invention relates to methods of administering Prostaglandin E1 (PGE1) and/or its synthetic analog Misoprotol to patients having CML.

Chronic myelogenous/myeloid leukemia (CML) is a cancer of the white blood cells. CML is a form of leukemia characterized by the increased and unregulated growth of predominantly myeloid cells in the bone marrow and the accumulation of these cells in the blood. Normal blood cells are all derived from hematopoietic stem cells (HSCs) with long-term (LT) or short-term (ST) blood reconstitution potentials in the bone marrow. The HSCs undergo multiple differentiation steps (including multipotent, oligopotent, and lineage-committed progenitors) and then give rise to all types of mature blood cells (See FIG. 1 for the hierarchical structure of HSCs, progenitors and blood cells). CML is a clonal bone marrow stem cell disorder caused by transformation of HSCs and/or various progenitors, which lead to massive production and proliferation of mature granulocytes (i.e., neutrophils, eosinophils, and basophils). These granulocytes are abnormal and do not develop into healthy white blood cells. Leukemia cells of CML accumulate in the blood and bone marrow allowing less room for the development of healthy white blood cells, red blood cells, and platelets, and as a result, infection, anemia, or easy bleeding may occur.

The Philadelphia chromosome (or the Philadelphia translocation) is a specific chromosomal abnormality that is associated with CML. The Philadelphia chromosome is the result of a reciprocal translocation between chromosome 9 and 22, and is specifically designated t(9;22)(q34;q11) (See FIG. 2). The presence of Philadelphia chromosome is a highly sensitive test for CML because 95% of people with CML have this abnormality. The translocation of the Philadelphia chromosome results in an oncogenic fusion between the BCR gene and the ABL gene, which encodes a tyrosine kinase. The gene product of the BCR-ABL fusion gene is a mutant form of the ABL tyrosine kinase called the “BCR-ABL tyrosine kinase,” which exhibits elevated kinase activity related to the wild-type ABL tyrosine kinase. Because the ABL tyrosine kinase activates a number of cell-cycle controlling proteins and enzymes, the elevated kinase activity of the BCR-ABL tyrosine kinase results in elevated cell proliferation. Moreover, the BCR-ABL tyrosine kinase inhibits gene repair which results in genomic instability and may cause a blast crisis in CML where CML begins to behave more like acute leukemia with rapid progression and short survival time.

Because of the recognized role of the BCR-ABL tyrosine kinase in CML, treatment of CML has largely focused on BCR-ABL tyrosine kinase inhibitors (TKIs). The first BCR-ABL tyrosine kinase inhibitor was imatinib (Gleevec®), which made a huge impact in the treatment of CML approximately 10 years ago. In 2010, the BCR-ABL tyrosine kinase inhibitors nilotinib and dasatinib also were approved for first-line therapy, making three drugs in this class available for treatment of newly diagnosed CML. TKI drugs are effective in inducing remissions and prolonging survival of CML patients at a chronic phase but are less effective against advanced phase CML. Disease recurrence is usually seen following cessation of drug treatment, even in patients with undetectable BCR-ABL expression by the most sensitive quantitative PCR method. As a result, most patients need to take the TKIs indefinitely, with risks of toxicity, drug resistance, and associated expense. The underlying reason is that leukemia is also organized in a hierarchical structure, with leukemic stem cells (LSCs) being responsible for continuous production of leukemic blast cells (See FIG. 3 for a conceptual illustration showing the comparison of the HSC-normal blood cell and LSC-leukemia cell hierarchies. The LSCs are not sensitive to the TM treatment and thus retained in patients achieving remission, being responsible for the replase. Collectively, a “cure” for CML is still elusive for most CML patients in spite of the success of TKI therapy. These issues indicate a need to identify co-therapy or therapeutic alternatives, particularly those having the capacity to eliminate CML LSCs.

### SUMMARY

The inventor has demonstrated that prostaglandin E1 (PGE1) and misoprostol may be utilized to treat CML patients. PGE1 and misoprostol may be utilized as a monotherapy or may be utilized in conjunction with other therapies for CML including BCR-ABL tyrosine kinase inhibitors.

Disclosed are methods and compositions for treating leukemia including chronic myelogenous/myeloid leukemia (CML). The methods typically include administering prostaglandin E1 (PGE1) or misoprostol to a CML patient. Optionally, the method may include further administering an inhibitor of BCR-ABL tyrosine kinase to the patient. For example, the methods may include administering PGE1 or misoprostol and optionally may include administering an inhibitor of BCR-ABL tyrosine kinase selected from the group consisting of imatinib, nilotinib, dasatinib, bosutinib, ponatinib, and bafetinib. In the disclosed methods, the prostaglandin E1 or misoprostol may be administered prior to, concurrently with, or after administering the inhibitor of BCR-ABL tyrosine kinase. Suitable doses of PGE1 (pharmaceutically known as Alprostadil) or misoprostol may include doses typically administered in clinics. PGE1 (Alprostadil) is used to treat erectile dysfunction and limb ischemia, and Misoprostol is used to treat stomach ulcers and postpartum bleeding. Because both PGE1 (Alprostadil) and Misoprostol are both FDA-approved drugs, the toxicity has be thoroughly tested. An optimal dose for treating CML (alone or in combination with TKIs) will be tested in clinical trials.

The compositions contemplated herein may include pharmaceutical compositions comprising PGE1 or Misoprostol and optionally comprising an inhibitor of BCR-ABL tyrosine kinase (e.g., an inhibitor selected from the group consisting of imatinib, nilotinib, dasatinib, bosutinib, ponatinib, and bafetinib). Also contemplated herein are kits that comprise a first pharmaceutical composition of PGE1 or Misoprostol and a second pharmaceutical composition of an inhibitor of BCR-ABL tyrosine kinase (e.g., an inhibitor selected from the group consisting of imatinib, nilotinib, dasatinib, bosutinib, ponatinib, and bafetinib).

### BRIEF DESCRIPTION OF THE FIGURES

FIG. 1 provides a chart illustrating the differentiation of various blood cells from a blood stem cell (namely HSCs). The long-term (LT) and short-term (ST) HSCs can give rise to multipotent progenitors (MPPs), which further differentiated into common myeloid progenitors (CMPs) and common lymphoid progenitors (CLPs). These progenitors generate different mature blood cells. Among these, CMPs and granulocyte-macrophage progenitors (GMPs) are precursors for granulocytes.

FIG. 2 illustrates the translocation event that results in the Philadelphia chromosome. The fusion protein, BCR-ABL, generated as a result of the translocation can transform HSCs, MPPs, CMPs, and GMPs, causing CML.

FIG. 3 demonstrates the concept of LSCs and the similarity between HSC-mature blood cell and LSC-leukemia cell hierarchies.

FIG. 4 provides the chemical structure of prostaglandin E1 (PGE1), PGE2 and Misoprostol. PGE1 and PGE2 are both 20-carbon fatty acid derivatives, structurally similar, with PGE2 containing an extra carbon-carbon double bond at the C5 position. Misoprostol is a synthetic PGE1, with modification of hydroxyl group location (from C15 to C16).

FIG. 5 shows the experimental design using a mouse model of CML followed by testing of therapeutic effects by PGE1 or PGE1, alone or in combination with one of TKIs, Imatinib. Bone marrow cells (BM) were isolated from donor mice, and HSCs were enriched and delivered the BCR-ABL gene using retroviral transduction. The BM cells were then transplanted into another group of recipient mice (called primary recipients here). The recipients will develop CML because the donor HSCs are transformed by BCR-ABL. On day 8 after BM transplantation (BMT), the primary recipients were treated with DMSO (the solvent to dissolve PGE1 or PGE2), PGE1, PGE2, Imatinib or both PGE1 and Imatinib. The survival of the recipients was monitored.

FIG. 6 demonstrates the effect of PGE1 monotherapy on the survival of CML recipients. CML was established as in FIG. 5. During days 8-36 post-bone marrow transplantation (BMT), the recipients were treated daily with 2.5 mg/kg body weight PGE1 or PGE2, or the vehicle DMSO. Statistical significance was determined by log-rank test. Data are pooled results from 2 independent experiments with a total of 10 CML recipients tested under each condition. Whereas all the recipients treated with DMSO and PGE1 succumbed to CML, PGE1 showed protective effect by itself.

FIG. 7 demonstrates the effect of PGE1 and Imatinib co-therapy on the survival of CML recipients. CML was established as in FIG. 5. During days 8-80 post-BMT, the recipients were treated daily with Imatinib alone (100 mg/kg body weight, b.i.d.) or in combination with PGE1 (2.5 mg/kg body weight). Statistical significance was determined by log-rank test. Data are pooled results from 2 independent experiments with a total of 9-10 CML recipients tested under each condition. Imatinib itself can substantially extend the survival of the CML recipients but all the recipients still died of the disease. On the other hand, the combination of Imatinib and PGE1 greatly extended the survival, with over 50% of the CML recipients protected at the end of observation period. Note that our further study on the mechanistic side, i.e., how PGE1 works provide additional insights. That is, PGE1 is more effective on LSCs and Imatinib is more effective in inhibiting the growth of leukemic blasts. This explains why the Imatinib +PGE1 combination is better than treatment with Imatinib alone or PGE1 alone.

FIG. 8 shows the experimental system to test the effect of PGE1 and Misoprostol on LSCs. The CML model was established in the primary recipients as in FIG. 5. The recipients were then treated with Imatinib, PGE1 or Misoprostol alone, or in combination during days 8-21 post-BMT. The LSCs were isolated from the BM of the primary recipients and transplanted into another set of secondary recipients. The ability of LSCs to propagate CML in the secondary recipients were tracked.

FIG. 9 demonstrates the effect of PGE1 or Misoprostol in reducing the ability of LSCs to propagate the CML leukemia. Both are superior to Imatinib treatment. Combination of Imatinib with PGE1 or Misoprostol can further enhance their favorable effect. Note that the LSCs were only treated with PGE1 or Misoprostol in the primary recipients for 2 weeks, and none of the secondary recipients were treated. These observations demonstrate the PGE1 and Misoprostol have long-lasting impact on LSCs, helping eradicate CML leukemia.

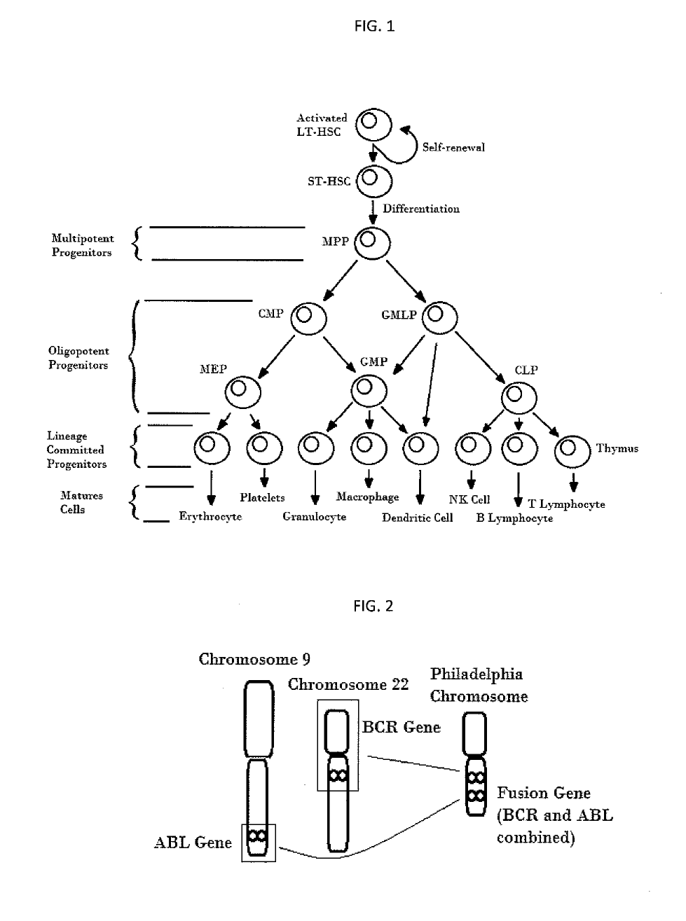

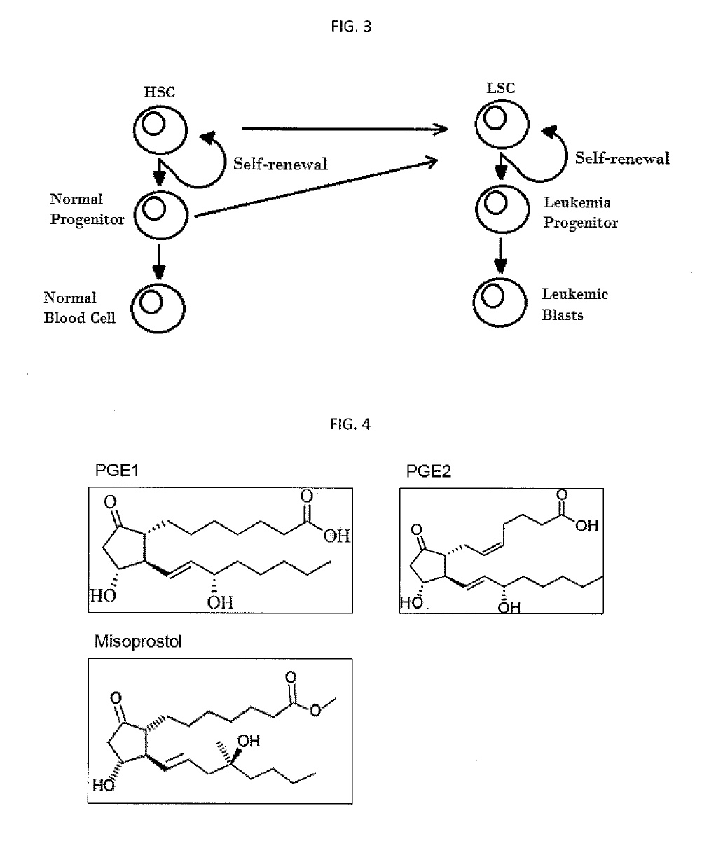

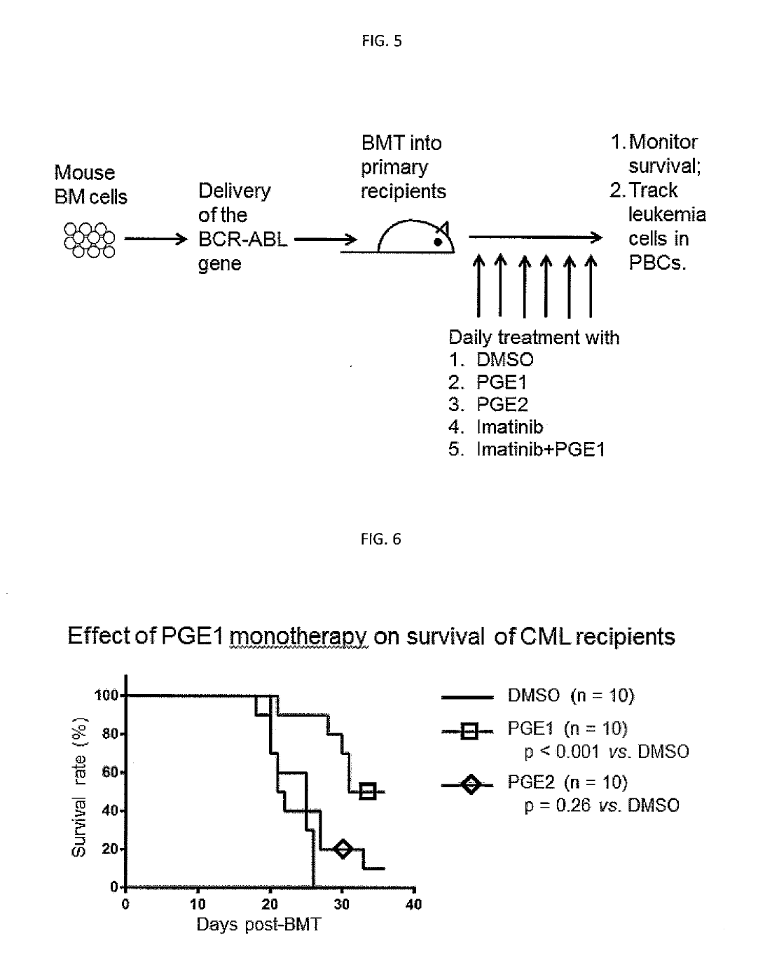

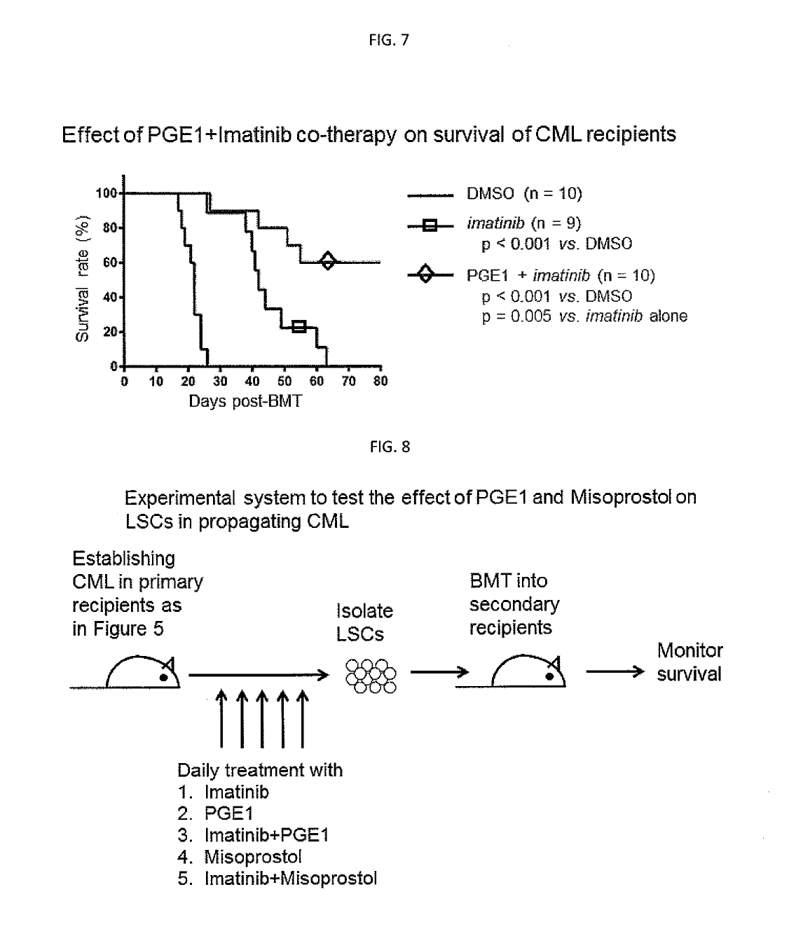

### DETAILED DESCRIPTION

The disclosed subject matter further may be described utilizing terms as defined below.

Unless otherwise specified or indicated by context, the terms “a”, “an”, and “the” mean “one or more.” For example, a “prostaglandin” should be interpreted to mean “one or more prostaglandins.” An “inhibitor” should be interpreted to mean “one or more inhibitors.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “patient” may be used interchangeably with the term “subject” or “individual” and may include an “animal” and in particular a “mammal.” Mammalian subjects may include humans and other non-human primates, domestic animals, farm animals, and companion animals such as dogs, cats, guinea pigs, rabbits, rats, mice, horses, cattle, cows, and the like.

A “patient in need thereof” is intended to include a patient having or at risk for developing chronic myelogenous/myeloid leukemia (CML). A “patient in need thereof” is intended to include a patient at risk for relapsing after having been treated for CML.

As is well known in the art, hematopoietic stem cells (HSCs) are adult stem cells that give rise to all mature blood cells in multiple lineages. These cells are derived from the mesoderm and are located in the red bone marrow contained in the core of most bones. HSCs have two distinct abilities: 1) self-renewal; and 2) differentiation. Leukemic stem cells (LSCs) generally give rise to bulk leukemia blasts and, like HSCs, also have the ability to self-renew and differentiate.

Tcf1 and Lef1 are proteins that behave as transcription factors. That is, they both have a conserved DNA-binding domain that recognizes a similar sequence. By binding to DNA, Tcf1 and Lef1 change gene transcription in HSCs and LSCs. The Inventor discovered the importance of Tcf1 and Lef1 as it pertains to the LSCs ability to self-renew. That is, for LSCs, lack of Tcf1 and Lef1 severely limits the ability for LSCs to reproduce. For HSCs, however, lack of Tef1 and Lef1 had only a minimal effect. For the sake of completeness, the Inventor's discovery resulted from a gene knockout approach wherein the Inventor deleted Tcf1 and Lef1 from LSCs and HSCs and subsequently studied the impact of their deletion.

In view of the Inventor's discovery, the Inventor set out to screen for drugs that would simulate the effect of eliminating Tcf1 and Lef1 for use in treatment of CML. Inventor experimented with the following drugs which are known to at least partly simulate the effect of eliminating Tcf1 and Lef1: Carbimazole, Cinchonine, Gibberellic acid, Hippeastrine hydrobromide, Dimaprit dihydrochloride, Spaglumic acid, and Vigabatrin. Unfortunately, these drugs had no effect on CML. Inventor further experimented with Trichostatin A and Vorinostat. Inventor discovered these latter drugs did have a strong effect on LSCs. However, because Trichostatin A and Vorinostat are toxic to HSCs, they presented little promise a practical drug usable for treating LSCs. Inventor thereafter turned to prostaglandins for treating CML.

Prostaglandin E2 (PGE2) is a naturally occurring prostaglandin (See FIG. 4 for chemical structure of PGE2). PGE2 is known to cause several biological effects such as inducing a fever, softening of a cervix, and causing uterine contractions. PGE2 also stimulates osteoblasts to release factors that stimulate resorption by osteoblasts. PGE2 is also a known vasodilator and an agent which may inhibit the release of noradrenaline from sympathetic new terminals.

Prostaglandin E1 (PGE1) is a potent endogenous vasodilator agent that increases peripheral blood flow. (See FIG. 4 for chemical structure of PGE1). PGE1 inhibits platelet aggregation and has many other biological effects such as bronchodilation, mediation of inflammation, and various protective functions. The protective action of PGE1 has been shown on both experimental animal models of liver injury and patients with fulminant viral hepatitis. Prostaglandin E1 is known pharmaceutically as alprostadil and has been approved by the FDA for the treatment of erectile dysfunction and has therapeutic vasodilatory properties. In healthy humans, prostaglandin E1 is biosynthesized “on demand” at its intended site of action from dihomo-γ-linolenic acid.

Prostaglandins including PGE1 have been described in the art as modulators of myeloid proliferation. (See, e.g., Taetle R, Mendelsohn J. Modulation of normal and abnormal myeloid progenitor proliferation by cyclic nucleotides and PGE1. Blood Cells. 1980;6(4):701-18; Aglietta M, Piacibello W, Gavosto F. Insensitivity of chronic myeloid leukemia cells to inhibition of growth by prostaglandin E1. Cancer Res. 1980 Jul;40(7):2507-11; Taetle R, Guittard J P, Mendelsohn J M. Abnormal modulation of granulocyte/macrophage progenitor proliferation by prostaglandin E in chronic myeloproliferative disorders. Exp Hematol. 1980 Nov;8 (10):1190-1201; Pelus L M, Gold E, Saletan S, Coleman M. Restoration of responsiveness of chronic myeloid leukemia granulocyte-macrophage colony-forming cells to growth regulation in vitro following preincubation with prostaglandin E. Blood. 1983 Jul;62(1):158-65; Cannistra S A, Herrmann F, Davis R, Nichols K, Griffin J D. Relationship between HLA-DR expression by normal myeloid progenitor cells and inhibition of colony growth by prostaglandin E. Implications for prostaglandin E resistance in chronic myeloid leukemia. J Clin Invest. 1986 Jan;77(1):13-20; and Aglietta M1, Piacibello W, Stacchini A, Sanavio F, Infelise V, Resegotti L, Gavosto F. Effect of interferon-gamma on HLA class II antigen expression and sensitivity to prostaglandin E1 by normal and leukemic myeloid progenitors. Leuk Res. 1988;12(4):299-303; the contents of which are incorporated herein by reference in their entireties).

The compositions disclosed and utilized herein may be formulated as pharmaceutical compositions for administration to a patient in need thereof. Such compositions can be formulated and/or administered in dosages and by techniques well known to those skilled in the medical arts taking into consideration such factors as the age, sex, weight, and condition of the particular patient, and the route of administration. The compositions may include pharmaceutically acceptable carriers, diluents, or excipients as known in the art. A “pharmaceutically acceptable” carrier, excipient, diluent, or stabilizer typically is not biologically or otherwise undesirable, i.e., the carrier, excipient, diluent, or stabilizer may be administered to a subject, along with a prostaglandin (e.g., PGE1) and/or an inhibitor of BCR-ABL tyrosine kinase (e.g., imatinib, nilotinib, dasatinib, bosutinib, ponatinib, and bafetinib). In some embodiments, the carrier, excipient, diluent, or stabilizer may be selected to minimize any degradation of the prostaglandin and/or the inhibitor of BCR-ABL tyrosine kinase or to minimize any adverse side effects in a patient to which the compositions are administered. Suitable carriers, excipients, or diluents for the compositions utilized in the disclosed methods may include, but are not limited to, fillers such as saccharides (e.g., lactose or sucrose, mannitol or sorbitol), cellulose preparations and/or calcium phosphates (e.g., tricalcium phosphate or calcium hydrogen phosphate), as well as binders (e.g., starch paste, using, for example, maize starch, wheat starch, rice starch, potato starch, gelatin, tragacanth, methyl cellulose, hydroxypropylmethylcellulose, sodium carboxy-methylcellulose, or polyvinyl pyrrolidone). Further, the compositions may include preservatives (e.g., anti-microbial or anti-bacterial agents such as benzalkonium chloride) or adjuvants.

The pharmaceutical composition disclosed herein may be delivered via a variety of routes including but not limited to intravenous, parenteral, (e.g., intradermal, intramuscular or subcutaneous delivery) and oral routes. Suitable formulations of the pharmaceutical compositions may include liquid formulations and/or solid formulations (e.g., powders).

### EXAMPLE

The following example is illustrative and should not be interpreted to limit the scope of the claimed subject matter.

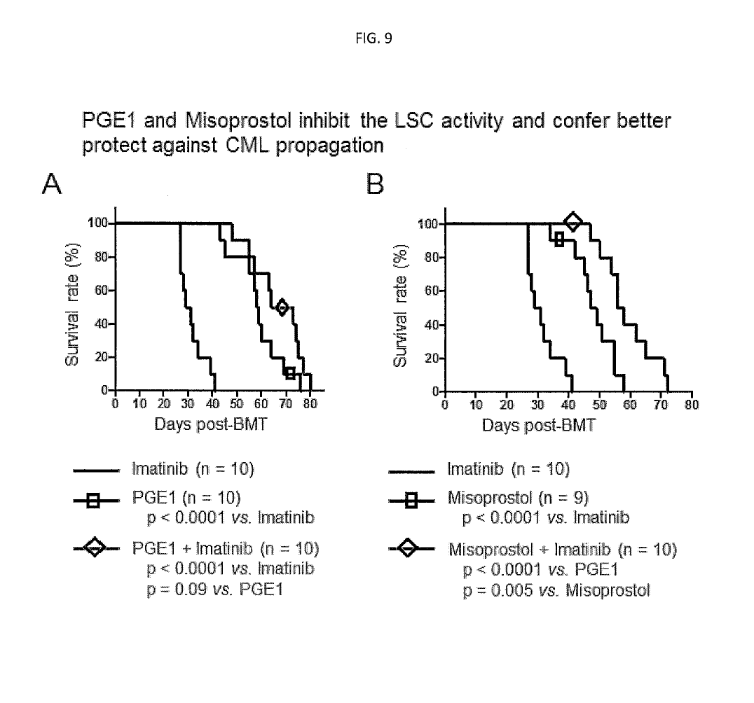

The present inventor has identified PGE1 and its synthetic analogue Misoprostol as a therapeutic for CML after performing a comparative transcriptome analysis of normal hematopoietic stem cells with those deficient for the Tcf1 and Lef1 transcription factors. The inventor has continued his studies by characterizing the in vivo effects of PGE1 in a CML mouse model, and demonstrated that treatment with PGE 1 alone was sufficient to significantly prolong the survival of CML mice. PGE2 was included in the test to demonstrate a specific beneficial effect for PGE1. CML animals were treated with the solvent dimethyl sulfoxide (DMSO) as a negative control (See FIG. 6). Results of testing showed the CML mice treated with PGE1 not only had an increase in survival, but an unexpectedly high survival rate when compared to the survival rate of PGE2-treated mice. For example, each of DMSO, PGE1, and PGE2 mice survived for approximately 17 days after bone marrow transplantation (BMT). However, beyond day 17 the survival rate of DMSO and PGE2 mice sharply declined such that by about day 22 nearly all DMSO-treated mice expired and by about day 35 the survival rate for the PGE2 mice was about 10%. However, the survival rate for PGE1 mice was significantly higher than either the DMSO mice or the PGE2 mice. For example, at about day 35 nearly 50% of the PGE1 mice had survived. In other words, the PGE1 mice had a nearly a 400% greater survival rate compared to the PGE2 mice, an outcome which was unexpected given the similarities between PGE1 and PGE2. The Inventor further demonstrated that the combination of PGE1 and Imatinib (one of the conventional TKIs used in CML therapy) is superior to the monotherapy by Imatinib alone (See FIG. 7). All the DMSO-treated CML mice died by day 26 after BMT. Imatinib extended the survival of CML mice, but they all eventually succumbed to the CML and died at approximately day 62 BMT. The treatment with both PGE1 and Imatinib further substantially extended the survival of CML mice, and most significantly, by day 80 BMT, 60% of the mice receiving the combination therapy had survived. The Inventor solidified this beneficial effect of treatment with PGE1 or Misoprostol by demonstrating that a two-week treatment had long-lasting impact on LSCs, substantially impaired the ability of LSCs to self-renew and to propagate the CML in new recipients (See FIG. 9). When the Imatinib-treated LSCs were transplanted into secondary CML recipients, the mice died at approximately day 40 after secondary BMT. By contrast, the PGE1- or Misoprostol-treated LSCs were much less efficient in propagating CML, with all the recipients survived beyond about day 60 BMT. Additionally, the combined treatment with PGE1 and Imatinib or Misoprostol and Imatinib showed better protective effect than mono-therapy with Imatinib, PGE1, or Misoprostol alone.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for treating chronic myelogenous/myeloid leukemia (CML) comprising:
administering prostaglandin E1 or a synthetic analog of prostaglandin E1 to a patient having chronic myelogenous/myeloid leukemia (CML).

2. The method of claim 1, further comprising:
administering an inhibitor of BCR-ABL tyrosine kinase to the patient.

3. The method of claim 2, wherein the prostaglandin E1 or the a synthetic analog of prostaglandin E1 is administered prior to administering the inhibitor of BCR-ABL tyrosine kinase.

4. The method of claim 2, wherein the prostaglandin E1 or the a synthetic analog of prostaglandin E1 is administered after administering the inhibitor of BCR-ABL tyrosine kinase.

5. The method of claim 2, wherein the prostaglandin E1 or the a synthetic analog of prostaglandin E1 is administered concurrently with the inhibitor of BCR-ABL tyrosine kinase.

6. The method of any of claims 2-5, wherein the inhibitor of BCR-ABL tyrosine kinase is selected from a group consisting of imatinib, nilotinib, dasatinib, bosutinib, ponatinib, and bafetinib.

7. A pharmaceutical composition comprising:
PGE1 or a synthetic analog of prostaglandin E1 and an inhibitor of BCR-ABL tyrosine kinase.

8. A kit comprising:
a first pharmaceutical composition comprising PGE1 or a synthetic analog of prostaglandin E1; and
a second pharmaceutical composition comprising an inhibitor of BCR-ABL tyrosine kinase.
